(19) United States
(12) Patent Application Publication
(10) Pub. No.: US 2026/0192025 A1
(43) Pub. Date: Jul. 9, 2026

(54) Breast Pump

(71) Applicant: Shenzhen Oceanwing Smart Innovations Technology Co., Ltd., Shenzhen (CN)

(72) Inventors: Shanshan Xiao, Shenzhen (CN); Zhihai Zhang, Shenzhen (CN); Liying Chen, Shenzhen (CN); Jianhua Zhong, Shenzhen (CN)

(21) Appl. No.: 19/457,321

(22) Filed: Jan. 23, 2026

Related U.S. Application Data
(63) Continuation of application No. PCT/CN2024/089571, filed on Apr. 24, 2024.

(30) Foreign Application Priority Data
Jul. 26, 2023 (CN) 202321994215.0
Jul. 28, 2023 (CN) 202310954976.1

(51) Int. Cl. A61M 1/06

(52) U.S. Cl. CPC A61M 1/066; A61M 2205/3653; A61M 2207/10

(57) ABSTRACT

The present application discloses a breast pump and a manufacturing method for the breast pump. The breast pump may comprise a soft rubber cover body and a heating element. The heating element may include a body portion and a connecting portion extending from the body portion. The soft rubber cover body may comprise a mounting hole on its outer wall. The body portion may be arranged in the soft rubber cover body, and the body portion may be adapted to the soft rubber cover body. The connecting portion may be arranged in the mounting hole, and a power port is exposed to the soft rubber cover body through the mounting hole.

### CROSS-REFERENCE TO RELATED APPLICATIONS

The present application claims priority to PCT/CN2024/089571, filed on Apr. 4, 2024, which claims all the benefits of the Chinese patent application No. 2023219942150, filed on Jul. 26, 2023 and entitled “Breast Pump Device”, and the Chinese patent application No. 2023109549761, filed on Jul. 28, 2023 and entitled “Breast Pump Accessory of Breast Pump and Manufacturing Method for Breast Pump Accessory of Breast Pump”. Each of the above applications is incorporated hereby in its entirety.

### FIELD

The present application relates to the technical field of maternal and child products, particularly to a breast pump, a breast pump accessory of the breast pump, and a manufacturing method for the breast pump accessory of the breast pump.

### BACKGROUND

Existing breast pumps on the market, mainly made of soft rubber, hard rubber, or soft and hard rubber, have no other functions except for breast suction. With the increasing demand for functionality, breast pumps are not only used for breast suction, but also need to have a series of functions such as hot compress, cold compress, and massage, while these functions are almost separate from the breast pumps.

In the related art, a plurality of instruments are often used together to achieve the desired effect, making the breast pumps more complex and heavier and consequently imposing greater burden on users during use.

### SUMMARY

The present application provides a breast pump, a breast pump accessory of the breast pump, and a manufacturing method for the breast pump accessory of the breast pump, to solve the problem that breast pumps in the related art cannot meet heating requirements.

One aspect of the present application provides a breast pump, comprising: a soft rubber cover body including a fluid channel; and a heating element including a body portion and a connecting portion extending from the body portion, where the connecting portion comprises a power port. The soft rubber cover body is provided with a mounting hole on its outer wall away from the fluid channel. The body portion is arranged in the soft rubber cover body, and the body portion is of a trumpet shape adapted to the soft rubber cover body. The connecting portion is arranged in the mounting hole, and the power port is exposed to the soft rubber cover body through the mounting hole. The soft rubber cover body is integrally formed with the heating element during injection molding, hydraulic forming, or silicone injection forming.

In an example, the body portion is formed by connecting side edges of a curved heating plate in an end-to-end manner, both the body portion and the connecting portion comprise through holes, and the through holes accommodate injection molding, hydraulic, or silicone injection sol; and a fixing plate is provided at the connection of the heating plate.

In an example, the body portion is formed by pressing a ring-shaped heating plate, both the body portion and the connecting portion are provided with through holes, and the through holes accommodate injection molding, hydraulic, or silicone injection sol.

In an example, the power port includes a plug socket, the plug socket is arranged in the mounting hole, the connecting portion is provided with a contact and a stud, the plug socket is fitted on the stud, and the contact is accommodated in the plug socket.

In an example, the power port further includes a plug cover plate, where the plug cover plate covers the plug socket and is provided with a spring pin, the plug socket is accommodated in the mounting hole, and the spring pin passes through the mounting hole and the plug socket and abuts against the contact.

In an example, the soft rubber cover body includes a first sub cover body and a second sub cover body that are fixedly connected. The body portion is arranged between the first sub cover body and the second sub cover body.

Another aspect of the present application provides a manufacturing method for a breast pump accessory of a breast pump, including: forming a first sub cover body and a second sub cover body through injection molding, hydraulic forming, or silicone injection forming, where the first sub cover body is retained in a first sub cover body mold and the second sub cover body is retained in a second sub cover body mold after mold opening; obtaining a heating element and placing the heating element in a preset position of the first sub cover body to obtain a pre-combination mold; combining the pre-combination mold and the second sub cover body mold; and obtaining the breast pump accessory of the breast pump after demolding.

In an example, the breast may further comprise a host, and a milk chamber assembly. The breast pump accessory includes a soft rubber cover body, a heating element, and a plug cover plate. The heating element is arranged in the soft rubber cover body. The plug cover plate is electrically connected to the heating element and penetrates through the soft rubber cover body. The host includes an electrical connection assembly, where the electrical connection assembly is electrically connected to the plug cover plate to supply power to the heating element. The host is provided with a first buckle portion on the periphery of the electrical connection assembly. The milk chamber assembly is arranged between the breast pump accessory and the host. The milk chamber assembly is provided with a connecting hole whose position corresponds to the plug cover plate, and the plug cover plate passes through the connecting hole and is connected to the electrical connection assembly. The milk chamber assembly is provided with a second buckle portion corresponding to the first buckle portion on the periphery of the connecting hole, and the second buckle portion is buckled with the first buckle portion.

Compared to the related art, the breast pump of the present application may include the soft rubber cover body and the heating element, where the soft rubber cover body includes the fluid channel, the heating element includes the body portion and the connecting portion extending from the body portion, and the connecting portion is provided with the power port connected to an external power supply to electrically heat the breast pump accessory. The soft rubber cover body may be provided with the mounting hole on its outer wall away from the fluid channel. The body portion may be arranged in the soft rubber cover body, the body portion may be frustum-shaped, and the body portion is adapted to the soft rubber cover body, ensuring that the heating element can be fully attached to the soft rubber cover body, and enhancing the heating capacity of the entire breast pump accessory and ensuring that the heat can be timely fed back to a mother body. The connecting portion may be arranged in the mounting hole, and the power port may be exposed to the soft rubber cover body through the mounting hole, so that the breast pump can be connected to an external power supply and continuously heated or insulated through the heating element. The soft rubber cover body may be integrally formed with the heating element during injection molding, hydraulic forming, or silicone injection forming, so that the heating element is more closely attached to the entire soft rubber cover body, and the heat of the heating element can be transferred with less loss to the position where the soft rubber cover body is attached to the mother body.

### BRIEF DESCRIPTION OF THE DRAWINGS

FIG. 1 is a schematic structural diagram of a breast pump accessory of a breast pump according to an example of the present application, where the breast pump accessory includes a heating element;

FIG. 2 is a partial cross-sectional structural diagram of the breast pump accessory of the breast pump in FIG. 1;

FIG. 3 is an enlarged structural diagram of circle A1 in the breast pump accessory of the breast pump in FIG. 2;

FIG. 4 is a schematic structural diagram of the heating element in FIG. 1;

FIG. 5 is a flowchart of a manufacturing method for a breast pump accessory of a breast pump according to an example of the present application, including steps S1, S2, S3, S4, and S5;

FIG. 6 is a flowchart of step S1 in FIG. 5 according to an example;

FIG. 7 is a flowchart of step S1 in FIG. 5 according to another example;

FIG. 8 is a flowchart of step S2 in FIG. 5 according to an example;

FIG. 9 is a flowchart of step S2 in FIG. 5 according to another example;

FIG. 10 is a flowchart of step S3 in FIG. 5 according to an example;

FIG. 11 is a flowchart of step S4 in FIG. 5 according to an example;

FIG. 12 is a flowchart of step S5 in FIG. 5 according to an example;

FIG. 13 is an exploded structural diagram of a breast pump according to an example of the present application;

FIG. 14 is a schematic structural diagram of the breast pump accessory shown in FIG. 1;

FIG. 15 is a schematic structural diagram of part B shown in FIG. 1;

FIG. 16 is a schematic structural diagram showing that a host faces one side of the breast pump accessory in FIG. 1;

FIG. 17 is a schematic structural diagram of part A shown in FIG. 1;

FIG. 18 is a cross-sectional structural diagram of the breast pump shown in FIG. 1;

FIG. 19 is a three-dimensional structural diagram of a plug cover plate shown in FIG. 1;

FIG. 20 is a schematic structural diagram of part D shown in FIG. 18;

FIG. 21 is a schematic structural diagram of part C shown in FIG. 18; and

FIG. 22 is another cross-sectional structural diagram of the breast pump shown in FIG. 1.

### DETAILED DESCRIPTION

The technical solutions in the present invention are clearly and completely described below in conjunction with the accompanying drawings. Apparently, the described examples are merely some of the examples of the present invention, not all of them. Based on the examples of the present invention, all other examples obtained by those of ordinary skill in the art without any creative efforts shall fall within the scope of protection of the present invention.

In the following description, specific details such as specific system structures, interfaces, and technologies are provided for illustration rather than limitation, to facilitate a thorough understanding of the present application.

The terms “system”, “unit”, and “network” are often used herein interchangeably. The term “and/or” herein describes only an association relationship for describing associated objects and represents that three relationships may exist. For example, A and/or B may represent the following three cases: only A exists, both A and B exist, and only B exists. In addition, the character “/” herein generally indicates an “or” relationship between contextually associated objects. In addition, the term “plurality” herein refers to two or more.

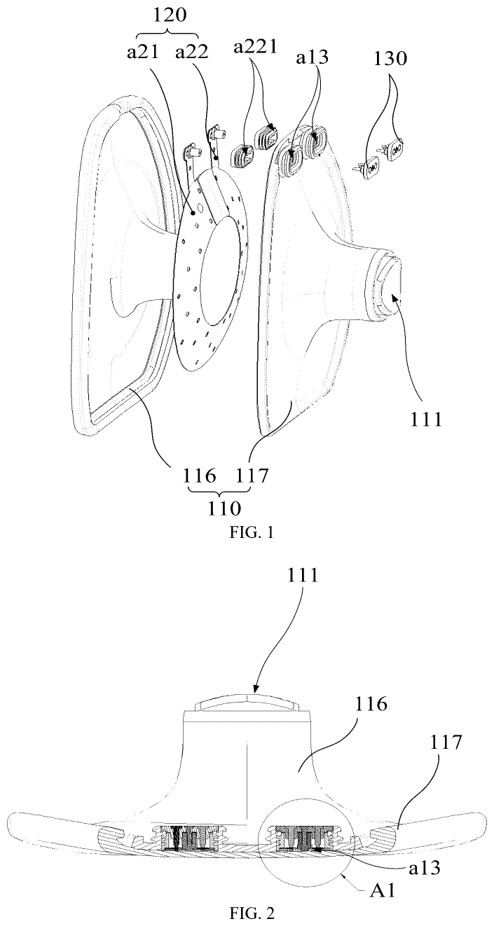

One aspect of the present application provides a breast pump accessory of a breast pump, as shown in FIG. 1. FIG. 1 is a schematic structural diagram of a breast pump accessory of a breast pump according to an example of the present application. The breast pump accessory of the breast pump includes a soft rubber cover body 110 and a heating element 120, where the soft rubber cover body 110 includes a fluid channel 111, and the soft rubber cover body 110 has smoother texture, good hydrophilic property, and better skin contact. The heating element 120 includes a body portion a21 and a connecting portion a22 extending from the body portion a21, where the connecting portion a22 is provided with a power port a221 connected to an external power supply (not shown) to electrically heat the breast pump accessory. The soft rubber cover body 110 is provided with a mounting hole a13 on its outer wall away from the fluid channel 111, the body portion a21 is arranged in the soft rubber cover body 110, the body portion a21 is frustum-shaped, and the body portion a21 is adapted to the soft rubber cover body 110 (e.g., has a 3D conical structure), ensuring that the heating element 120 can be fully attached to the soft rubber cover body 110, enhancing the heating capacity of the entire breast pump accessory and ensuring timely feedback of heat to the mother body. The body portion a21 may be adapted to a shape of the soft rubber cover body 110.

The body portion a21 may be frustum-shaped, as long as the body portion a21 can adapt to the soft rubber cover body 110 to achieve the attachment of the heating element 120 to the soft rubber cover body 110. Without affecting the heating effect of the entire breast pump accessory, the body portion a21 may be designed in a desired shape according to the actual needs of the breast pump accessory. The connecting portion a22 is arranged in the mounting hole a13, and the power port a221 is exposed to the soft rubber cover body 110 through the mounting hole a13, so that the breast pump accessory of the breast pump can be connected to the external power supply and continuously heat or insulate the breast pump through the heating element 120. The soft rubber cover body 110 is integrally formed with the heating element 120 during injection molding, hydraulic forming, or silicone injection forming, so that the heating element 120 is more closely attached to the entire soft rubber cover body 110, and the heat of the heating element 120 can be transferred with less loss to the position where the soft rubber cover body 110 is attached to the mother body.

FIG. 4 is a schematic structural diagram of the heating element in FIG. 1. In some examples, the body portion a21 is formed by connecting side edges of a curved heating plate (not shown) in an end-to-end manner. In order to ensure the stability of fixation, both the body portion a21 and the connecting portion a22 are provided with through holes a210. When the soft rubber cover body 110 and the heating element 120 are jointly subjected to injection molding, hydraulic forming, or silicone injection forming, the through holes a210 accommodate injection molding, hydraulic, or silicone injection sol (e.g., gel), to further ensure the tightness of fixation between the soft rubber cover body 110 and the heating element 120. A fixing plate a20 is provided at the connection of the heating plate to fix the shape of the heating element 120, ensuring the stability of the three-dimensional structure of the heating element 120 and the effectiveness of the breast pump accessory of the breast pump.

In some examples, the body portion a21 is formed by directly pressing a ring-shaped heating plate, with a conical structure that has the same radian as the soft rubber cover body 110. In this case, the heating element 120 does not need to be further treated, but the heating element 120 and the soft rubber cover body 110 are cooperatively connected and tightly connected through injection molding, hydraulic forming, or silicone injection to form the breast pump accessory of the breast pump.

With continued reference to FIG. 1, in some examples, the soft rubber cover body 110 includes a first sub cover body 117 and a second sub cover body 116, where the first sub cover body 117 and the second sub cover body 116 are fixedly connected by injection molding, hydraulic forming, or silicone injection. Without affecting the tightness of the entire soft rubber cover body 110, the heating element 120 may be accommodated in the soft rubber cover body 110. The body portion a21 is arranged between the first sub cover body 117 and the second sub cover body 116, to achieve uniform heating of the soft rubber cover body 110, thereby further improving the heating usage experience of the breast pump accessory of the breast pump.

In an example, in order to ensure that the breast pump accessory of the breast pump reaches an appropriate temperature during heating and can be powered off or insulated in a timely manner. As shown in FIG. 1 and FIG. 4, in some examples, either the soft rubber cover body 110 or the body portion a21 is provided with a temperature sensor a15 to detect the temperature of the entire heating element 120, ensuring effective heating temperature and avoiding overheating. A fixing element (not shown) is provided at an end of the temperature sensor a15 away from the heating element 120, to ensure that the temperature sensor a15 can still be stably fixed in its original position when the heating element 120 is bent.

Further, a protective ring (not shown) is provided at the other end of the temperature sensor a15. The combined action of the protective ring and the fixing element at both ends of the temperature sensor a15 further stabilizes the position of the temperature sensor a15, ensuring the stability of the heated temperature of the breast pump accessory and avoiding burns to the mother body due to excessive temperature during use.

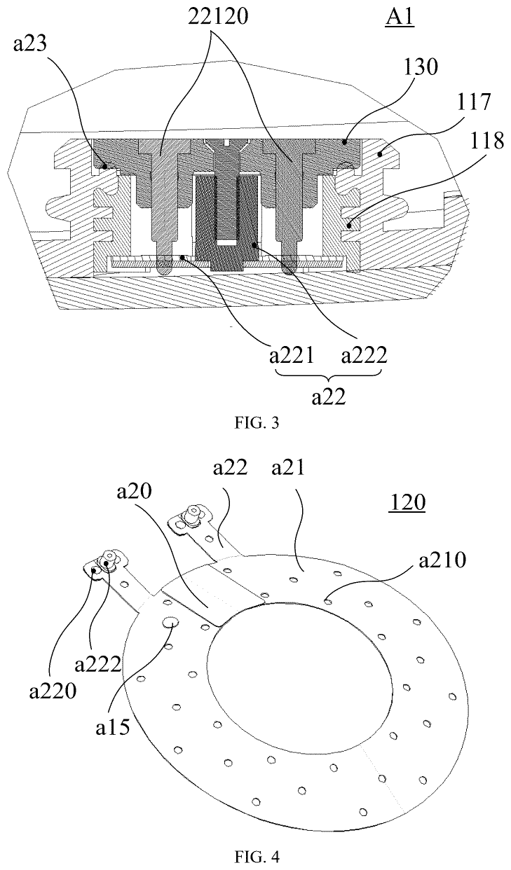

Refer to FIG. 2 and FIG. 3. FIG. 2 is a partial cross-sectional structural diagram of the breast pump accessory of the breast pump in FIG. 1, and FIG. 3 is an enlarged structural diagram of circle A1 in the breast pump accessory of the breast pump in FIG. 2. In some examples, the power port a221 includes a plug socket 118, the plug socket 118 is arranged in the mounting hole a13. The heating element 120 passes through the soft rubber cover body 110 and is connected to the external power supply for heating. The connecting portion a22 is provided with a contact a220 and a stud a222, the stud a222 is used to accommodate and fix a screw. The plug socket 118 is fitted on the stud a222, and the contact a220 is accommodated in the plug socket 118, preventing the contact a220 from contacting the injection molding, hydraulic, or silicone injection sol during injection molding, hydraulic forming, or silicone injection to affect its sensitivity and further affect the degree of heating of the breast pump accessory. The contacts a220 are positive and negative electrodes of the heating element 120 and the temperature sensor a15, respectively.

Understandably, a rubber groove (not shown) is provided at the contact position between the power port a221 and the soft rubber cover body 110. When the soft rubber cover body 110 is subjected to injection molding, hydraulic forming, or silicone injection, the rubber groove on the power port a221 is also filled with the injection molding, hydraulic, or silicone injection sol, thereby increasing the contact area between the soft rubber cover body 110 and the power port a221, and further ensuring the structural stability of the breast pump accessory of the entire breast pump.

Further, the power port a221 includes a plug cover plate 130, which serves as a medium connected to the external power supply. The plug cover plate 130 covers the plug socket 118, the plug cover plate 130 is provided with a fixing hole, and a fixing screw passes through the fixing hole and the stud a222 to fix the plug cover plate 130 to the power port a221, avoiding disconnection or short-circuiting due to unstable connection between the power port a221 and the plug cover plate 130 when the plug cover plate 130 is connected to the external power supply, and further avoiding danger. The plug cover plate 130 is further provided with a spring pin 22120, the plug socket 118 is accommodated in the mounting hole a13, and the spring pin 22120 passes through the mounting hole a13 and the plug socket 118 and abuts against the contact a220, to connect the plug cover plate 130 and the heating element 120, ensuring the stability of current and heating of the heating element 120 when connected to the external power supply, and further enhancing the user experience of the heating mode of the breast pump accessory.

Understandably, a contact reinforcement plate (not shown) is further provided around the contact a220. The spring pin 22120, when fixed to the contact a220, abuts against the contact reinforcement plate, to avoid poor contact due to excessively small contact area between the contact a220 and the spring pin 22120 and disengagement of the spring pin 22120 from the contact position of the contact a220 when the breast pump shakes or moves.

The plug cover plate 130 and the plug socket 118 may alternatively be fixed by a series of bonding methods such as gluing, fastening, and ultrasonic treatment. A sealing element a23 may further be provided at the connection between the plug cover plate 130 and the plug socket 118, to prevent water from entering the interior of the breast pump accessory from the gap during cleaning to affect normal operation.

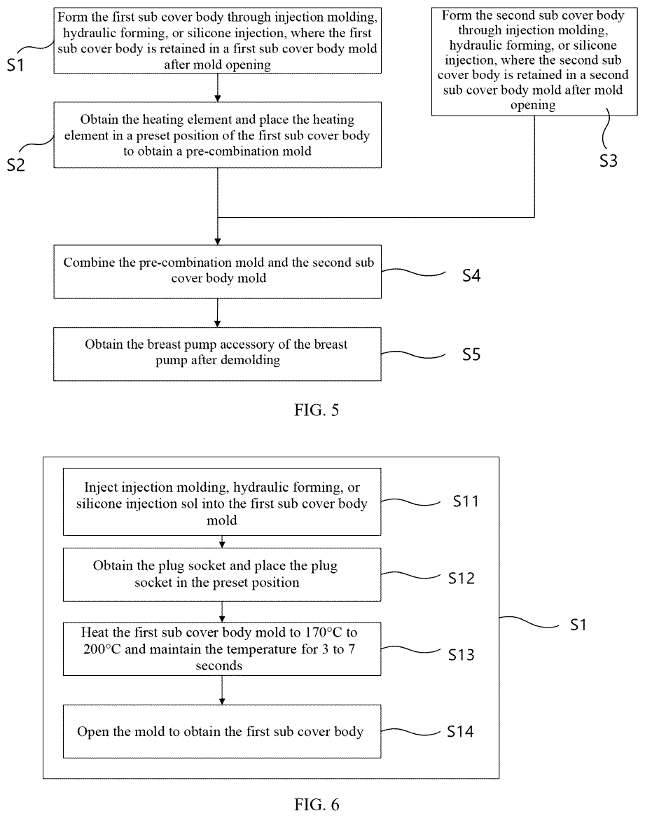

Another aspect of the present application proposes a manufacturing method for a breast pump accessory of a breast pump, as shown in FIG. 5. FIG. 5 is a flowchart of a manufacturing method for a breast pump accessory of a breast pump according to an example of the present application. The manufacturing method may include:
Step S1: Form the first sub cover body 117 through injection molding, hydraulic forming, or silicone injection, where the first sub cover body 117 is retained in a first sub cover body mold after mold opening. When injection molding, hydraulic forming, or silicone injection is performed to form the breast pump accessory of the entire breast pump, injection molding, hydraulic, or silicone injection sol is injected into the first sub cover body mold, which is then treated. When the first sub cover body 117 reaches a desired state, the first sub cover body 117 is released from the mold for later use.

The first sub cover body mold may be divided into an upper operating position and a lower operating position. When injection molding, hydraulic forming, or silicone injection is performed on the first sub cover body mold, the upper operating position and the lower operating position are combined and subjected to injection molding, hydraulic forming, or silicone injection. When the first sub cover body 117 reaches the desired state, the first sub cover body 117 is released from the upper operating position, and a portion of the first sub cover body 117 is retained in the lower operating position for later use.
Step S2: Obtain the heating element 120 and place the heating element 120 at a preset position of the first sub cover body 117 to obtain a pre-combination mold. The formed heating element 120 is placed at the preset position of the first sub cover body 117, facilitating the attachment of the heating element 120 to the soft rubber cover body 110, making the heating effect of the breast pump accessory more uniform, and improving user experience. By preventing the heating element 120 on the first sub cover body 117, the heating element 120 is also accommodated in the lower operating position of the first sub cover body mold, to form the pre-combination mold.
Step S3: Form the second sub cover body 116 through injection molding, hydraulic forming, or silicone injection, where the second sub cover body 116 is retained in a second sub cover body mold after mold opening. When injection molding, hydraulic forming, or silicone injection is performed to form the breast pump accessory of the entire breast pump, injection molding, hydraulic, or silicone injection sol is injected into the second sub cover body mold, which is then treated. When the second sub cover body 116 reaches a desired state, the second sub cover body 116 is released from the mold for later use.

The second sub cover body mold may also be divided into an upper operating position and a lower operating position. When injection molding, hydraulic forming, or silicone injection is performed on the second sub cover body mold, the upper operating position and the lower operating position are combined and subjected to injection molding, hydraulic forming, or silicone injection. When the second sub cover body 116 reaches the desired state, the second sub cover body 116 is released from the lower operating position, and a portion of the second sub cover body 116 is retained in the upper operating position for later use.

Further, in some implementation scenarios, the injection molding, hydraulic forming, or silicone injection forming is performed on the first sub cover body 117 and the second sub cover body 116 synchronously, to reduce processing time costs.

Understandably, step S1 and step S3 are not in order, the first sub cover body 117 and the second sub cover body 116 may be formed simultaneously, or step S1 or step S3 may be prioritized according to the requirements.
Step S4: Combine the pre-combination mold and the second sub cover body mold. In some implementation scenarios, after the first sub cover body 117 and the second sub cover body 116 are formed separately, the lower operating position of the first sub cover body mold and the upper operating position of the second sub cover body mold are combined for further treatment, and the fully formed breast pump accessory is cooled and released from the combined mold.
Step S5: Obtain the breast pump accessory of the breast pump after demolding. The released breast pump accessory of soft rubber with the heating element needs to be assembled with other structures to form a complete breast pump.

In order to ensure that the heating element 120 does not shift when the heating element 120 is placed in the preset position of the first sub cover body 117, the first sub cover body 117 needs to be maintained in its semi-cured state during injection molding, hydraulic forming, or silicone injection molding, as shown in FIG. 6. FIG. 6 is a flowchart of step S1 in FIG. 5 according to an example. Step S1 may include:
Step S11: Inject injection molding, hydraulic forming, or silicone injection sol into the first sub cover body mold. In some specific implementation scenarios, the injection molding, hydraulic forming, or silicone injection sol of soft rubber is injected into the first sub cover body mold, where the soft rubber has certain elasticity, better fluidity, and closer fit to the skin.
Step S12: Obtain the plug socket 1181 and place the plug socket 118 in the preset position. In some implementation scenarios, the connection between the heating element 120 and the external power supply, e.g., the plug socket 118, is reserved before the first sub cover body 117 is formed. A mold for the plug socket 118 is placed in a suitable position inside the first sub cover body mold and fixed.

Moreover, in other implementation scenarios, the first sub cover body mold may be directly designed to have the shape of the mold for the plug socket 118, and the plug socket 118 and the first sub cover body 117 may be integrally formed through injection molding, hydraulic forming, or silicone injection molding in the separate first sub cover body mold, reducing the forming steps of the first sub cover body 117, simplifying manual operation, and reducing labor costs.
Step S13: Heat the first sub cover body mold to 170° C. to 200° C. and maintain the temperature for 3 to 7 seconds. In this implementation scenario, the first sub cover body 117 is in a semi-cured state of the injection molding, hydraulic forming, or silicone injection sol, and the first sub cover body mold may be heated to 170° C., 180° C., 190° C., or 200° C., and maintained for appropriate time, such as 3 seconds, 5 seconds, or 7 seconds. As such, the first sub cover body 117 has certain hardness and fluidity, which can ensure that the heating element 120 can still be in a centered state between the first sub cover body 117 and the second sub cover body 116, avoiding deviation.

In an example, the first sub cover body mold is heated to 190° C. and maintained for 5 seconds to maintain the first sub cover body 117 in the semi-cured state.

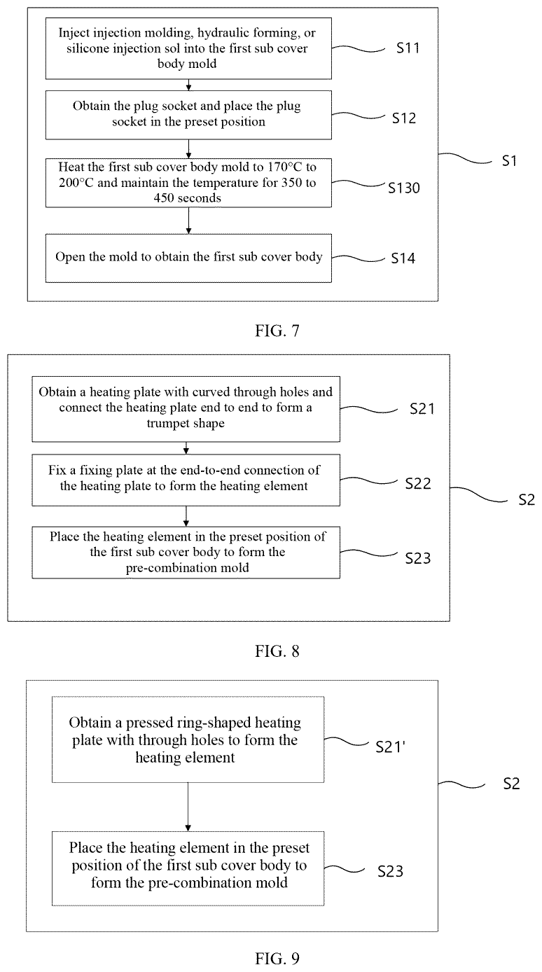

In other implementation scenarios, refer to FIG. 7. FIG. 7 is a flowchart of step S1 in FIG. 5 according to another example. The injection molding, hydraulic forming, or silicone injection molding of the first sub cover body 117 may alternatively include other steps:
Step S130: Heat the first sub cover body mold to 170° C. to 200° C. and maintain the temperature for 350 to 450 seconds. In this implementation scenario, when a structure capable of being used for fixing the heating element 120 is provided in the first sub cover body mold, the first sub cover body 117 may be directly heated to a fully cured state to stabilize its state. Specifically, the first sub cover body mold may be heated to 170° C., 180° C., 190° C., or 200° C., and maintained for appropriate time, such as 350 seconds, 370 seconds, 390 seconds, 400 seconds, 420 seconds, 440 seconds, or 450 seconds. The fixing structure ensures the stability of the heating element 120 when the first sub cover body mold and the second sub cover body mold are combined.

In an example, the first sub cover body mold may be heated to 190° C. and maintained at this temperature for 400 seconds to maintain the first sub cover body 117 in a stable state.
Step S14: Open the mold to obtain the first sub cover body 117. In this implementation scenario, when the first sub cover body 117 is formed or reaches the desired state, the upper operating position of the first sub cover body mold is moved away from the first sub cover body mold, and the first sub cover body 117 is accommodated in the lower operating position.

In order to better ensure that the heating element 120 can be tightly attached to the soft rubber cover body 110, the heating element 120 can be preferentially bent or preset, as shown in FIG. 8. FIG. 8 is a flowchart of step S2 in FIG. 5 according to an example. Step S2 may include:
Step S21: Obtain a heating plate with curved through holes and connect the heating plate end to end to form a trumpet shape. In this implementation scenario, the heating plate is sector-shaped. Generatrices of the heating plate are connected end to end to form a trumpet-shaped frustum-like surface, and the trumpet-shaped heating plate is attached to the first sub cover body 117. Understandably, in order to further ensure the attachment between the heating element 120 and the first sub cover body 117, the central angle of the heating plate should be the same as the central angle of the soft rubber cover body 110.

Understandably, in some examples, the heating plate may be metallic or non-metallic, and a suitable heating plate material may be selected according to the temperature design requirements and actual design structures of different breast pumps, to meet various usage requirements.
Step S22: Fix a fixing plate a20 at the end-to-end connection of the heating plate to form the heating element 120. In this implementation scenario, the mold closing of the first sub cover body 117 and the second sub cover body 116 requires high temperature and long maintenance time. Whether the heating plate is welded end to end or connected through other fixing elements under prolonged high temperature adjustment, a fusing phenomenon may occur. Therefore, the fixing plate a20 is provided at the end-to-end connection. The fixing plate a20 is fixed to the heating element 120 by fastening bolts, to further enhance the stability of the heating element 120.

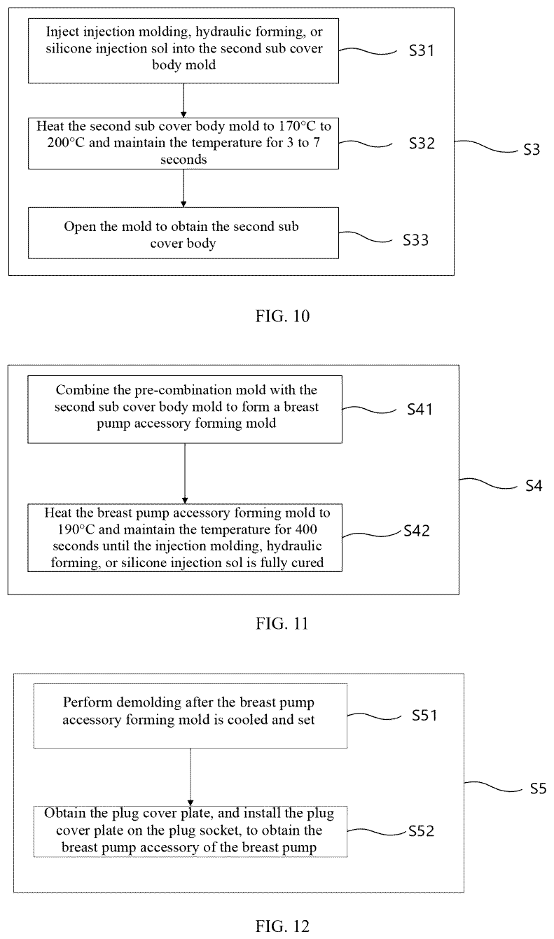

In other implementation scenarios, refer to FIG. 9. FIG. 9 is a flowchart of step S2 in FIG. 5 according to another example. The heating element 120 may be obtained by other step:
Step S21′: Obtain a pressed ring-shaped heating plate with through holes to form the heating element. The heating element 120 may be directly pressed into a conical structure having the same radians) as the soft rubber cover body 110, to further save installation steps and improve installation efficiency.
Step S23: Place the heating element 120 in the preset position of the first sub cover body 117 to form the pre-combination mold. The heating element 120, which is bent and has the same radian as the first sub cover body 117, is attached to the first sub cover body 117 to form the pre-combination mold for later use. Similarly, when the second sub cover body 116 is formed by injection molding, hydraulic forming, or silicone injection, in order to ensure that the first sub cover body 117 and the second sub cover body 116 can be completely attached to each other in the mold, the second sub cover body 116 is usually also formed in a semi-cured state, as shown in FIG. 10. FIG. 10 is a flowchart of step S3 in FIG. 5 according to an example. Step S3 may include:
Step S31: Inject injection molding, hydraulic forming, or silicone injection sol into the second sub cover body mold. In some specific implementation scenarios, the injection molding, hydraulic forming, or silicone injection sol of soft rubber is injected into the second sub cover body mold. In order to ensure that the first sub cover body 117 and the second sub cover body 116 can be completely fused and attached to each other, the first sub cover body 117 and the second sub cover body 116 should be made of the same material.
Step S32: Heat the second sub cover body mold to 170° C. to 200° C. and maintain the temperature for 3 to 7 seconds. In this implementation scenario, the second sub cover body 116 is in a semi-cured state of the injection molding, hydraulic forming, or silicone injection sol. As such, the second sub cover body 116 has certain hardness and fluidity. Specifically, the second sub cover body mold may be heated to 170° C., 180° C., 190° C., or 200° C., and maintained for appropriate time, such as 3 seconds, 5 seconds, or 7 seconds. When the lower operating position of the second sub cover body mold is moved away, the second sub cover body 116 can still remain in the upper operating position of the second sub cover body mold and has certain stability.

In an example, the second sub cover body mold is heated to 190° C. and maintained at this temperature for 5 seconds to maintain the second sub cover body 116 in a stable state.
Step S33: Open the mold to obtain the second sub cover body 116. At this time, the second sub cover body 116 is located at the upper operating position of the second sub cover body mold, and is to be combined with the first sub cover body 117 to form the overall structure of the breast pump accessory.

Refer to FIG. 11. FIG. 11 is a flowchart of step S4 in FIG. 5 according to an example. Step S4 may include:
Step S41: Combine the pre-combination mold with the second sub cover body mold to form a breast pump accessory forming mold. In this implementation scenario, the pre-combination mold where the first sub cover body 117 is located and the second sub cover body mold where the second sub cover body 116 is located are arranged at the same position through operation, and are combined to fix the first sub cover body 117 and the second sub cover body 116 together.
Step S42: Heat the breast pump accessory forming mold to 170° C. to 200° C. and maintain the temperature for 350 to 450 seconds until the injection molding, hydraulic forming, or silicone injection sol is fully cured. When the first sub cover body 117 and the second sub cover body 116 are combined, the two need to be fused and connected. Specifically, the first sub cover body mold and the second sub cover body mold may be heated to 170° C., 180° C., 190° C., or 200° C., and maintained for appropriate time, such as 350 seconds, 370 seconds, 390 seconds, 400 seconds, 420 seconds, 440 seconds, or 450 seconds. At least edge areas of the first sub cover body 117 and the second sub cover body 116 need to be melted and connected together, so that the entire soft rubber cover body 110 is formed as a whole.

In an example, the first sub cover body mold and the second sub cover body mold are heated together to 190° C. and maintained at this temperature for 400 seconds to maintain the formed soft rubber cover body 110 in a stable state.

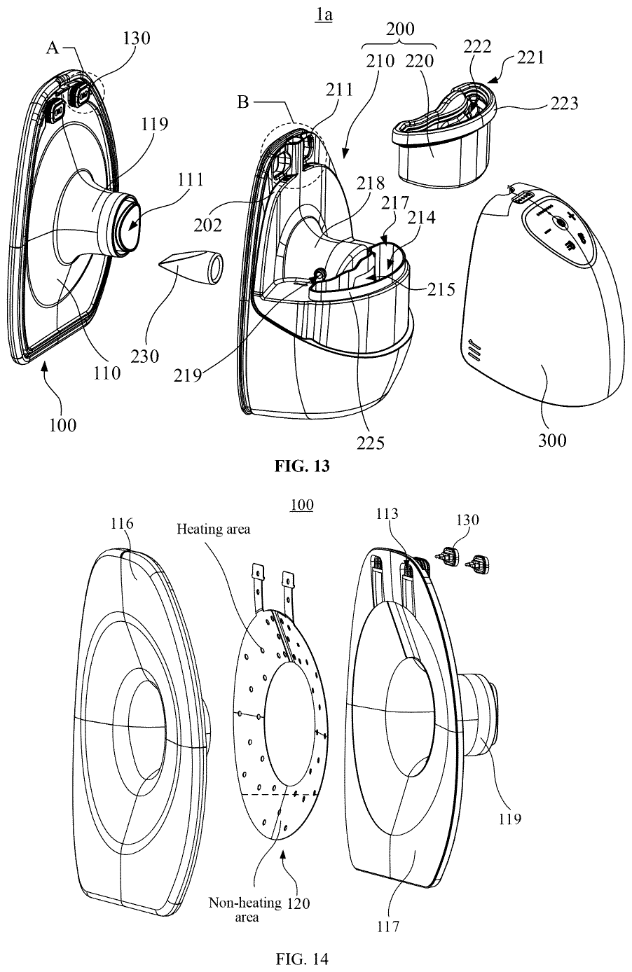

In some implementation scenarios, after the injection molding, hydraulic pressure, or silicone injection is completed, the medium connected to the external power supply needs to be assembled on the breast pump accessory, as shown in FIG. 12. FIG. 12 is a flowchart of step S5 in FIG. 5 according to an example. Step S5 may include:
Step S51: Perform demolding after the breast pump accessory forming mold is cooled and set. After the first sub cover body 117 and the second sub cover body 116 are combined and the soft rubber cover body 110 formed by the first sub cover body 117 and the second sub cover body 116 is completely cooled and set, a breast pump accessory structure formed by injection molding, hydraulic forming, or silicone injection molding is obtained by demolding.
Step S52: Obtain the plug cover plate 130, and install the plug cover plate 130 on the plug socket 118, to obtain the breast pump accessory of the breast pump. In this implementation scenario, the plug cover plate 130 covers the plug socket 118, and the plug cover plate 130 is fastened to the plug socket 118 through fixing screws, to avoid unstable connection between the breast pump accessory and the external power supply due to poor contact between the plug cover plate 130 and the plug socket 118, ensuring user experience.

Further, the inventor has found through long-term research that the emergence of breast pumps has made breastfeeding more convenient. The breast pump is used to extract breast milk and store the breast milk in a milk chamber assembly, making it convenient for infants to suck when needed. The breast pump may be provided with a heating element for heating. Currently, in the structure settings of breast pumps in the related art, water short-circuiting in the heating element and unstable connection between the heating element and the power supply affect the stability of the heating function of the breast pumps. To solve the technical problem, the present application provides the following examples.

As shown in FIG. 13 to FIG. 16, the breast pump 1a described in the present application may include a breast pump accessory 100, a host 300, and a milk chamber assembly 200. The breast pump accessory 100 may include the soft rubber cover body 110, the heating element 120, and the plug cover plate 130. The heating element 120 is arranged inside the soft rubber cover body 110. The plug cover plate 130 is electrically connected to the heating element 120 and penetrates through the soft rubber cover body 110. The host 300 includes an electrical connection assembly 310, where the electrical connection assembly 310 is electrically connected to the plug cover plate 130 to supply power to the heating element 120. The host 300 is provided with a first buckle portion 304 on the periphery of the electrical connection assembly 310. The milk chamber assembly 200 is arranged between the breast pump accessory 100 and the host 300. The milk chamber assembly 200 is provided with a connecting hole 211 whose position corresponds to the plug cover plate 130, and the plug cover plate 130 passes through the connecting hole 211 and is connected to the electrical connection assembly 310. The milk chamber assembly 200 is provided with a second buckle portion 201 corresponding to the first buckle portion 304 on the periphery of the connecting hole 211, and the second buckle portion 201 is buckled with the first buckle portion 304.

The breast pump 1a is used to extract breast milk and store the breast milk in the milk chamber assembly 200, making it convenient for infants to suck when needed. The soft rubber cover body 110 is provided with a fluid channel 111. During the use of the breast pump 1a, one side of the soft rubber cover body 110 can be in contact with the human skin, allowing breast milk to enter the fluid channel 111 through the end close to the human body through the fluid channel 111 and then enter the milk chamber body assembly 200. Further, the fluid channel 111 has a trumpet-shaped opening at the end facing the human body, to better fit the human body. In some examples, the host 300 is provided with a suction device 316. The suction device 316 generates negative pressure in the fluid channel 111 during operation, thereby sucking breast milk into the fluid channel 111.

The heating element 120 is used to heat the soft rubber cover body 110, to promote human lactation and improve the comfort of a user wearing the breast pump 1a. The host 300 can supply power to the heating element 120. For example, the host 300 may be provided with an energy storage assembly, which is used to supply power to the heating element 120. In an example, the energy storage assembly may be electrically connected to the electrical connection assembly 310, and electrically connected to the heating element 120 through the electrical connection assembly 310 and the plug cover plate 130 that are electrically connected to each other, thereby supplying power to the heating element 120. The host 300 may alternatively be connected to a power socket, to supply power to the heating element 120.

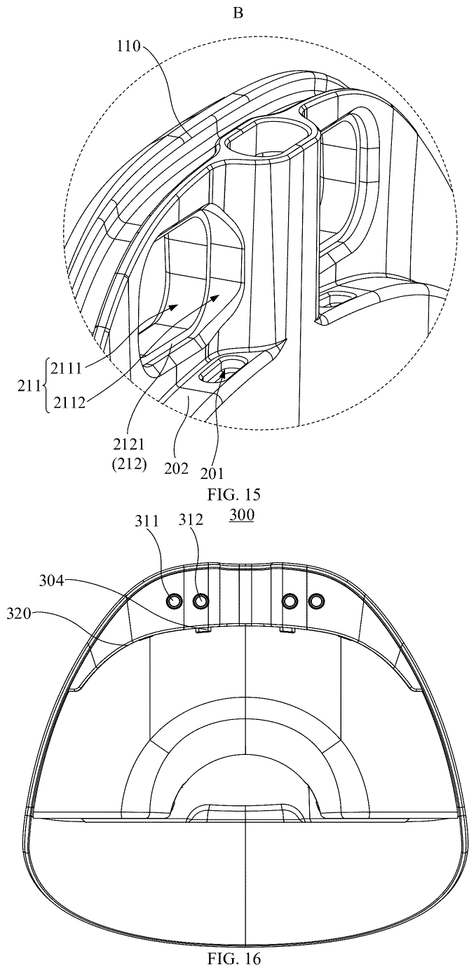

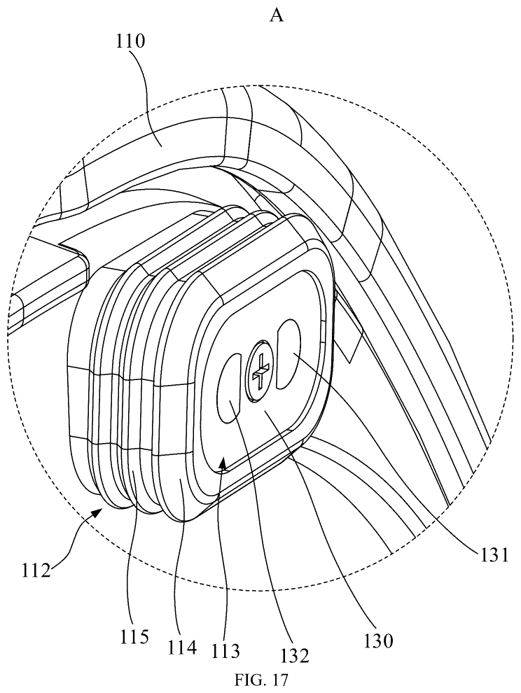

As shown in FIG. 15, FIG. 16, and FIG. 20, the second buckle portion 201 and the first buckle portion 304 maintain the milk chamber assembly 200 and the host 300 relatively fixed on the periphery of the connecting hole 211, facilitating stable connection between the electrical connection assembly 310 and the plug cover plate 130.

In an example, as shown in FIG. 13, one, two, or more mounting portions 112 may be designed. The connecting hole 211, the plug cover plate 130, and the electrical connection assembly 310 are identical in quantity to and correspond one to one with the mounting portion 112. The second buckle portion 201 is correspondingly provided on the periphery of each connecting hole 211, promoting stable connection between each plug cover plate 130 and the corresponding electrical connection assembly 310.

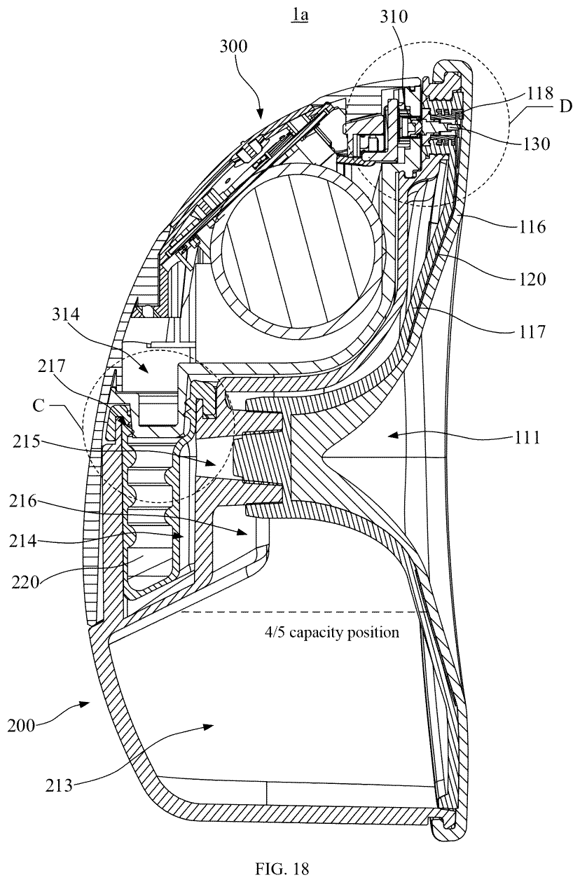

In an example, as shown in FIG. 18 and FIG. 20, the buckling direction of the second buckle portion 201 and the first buckle portion 304 intersect with a penetration direction of the connecting hole 211.

As such, the buckling connection between the second buckle portion 201 and the first buckle portion 304 can limit the relative movement of the milk chamber assembly 200 and the host 300 in the penetration direction of the connecting hole 211, thereby limiting the movement of the milk chamber assembly 200 and the host 300 away from each other.

When the breast pump 1a is assembled, the breast pump accessory 100 may be assembled to the milk chamber assembly 200 along the penetration direction of the connecting hole 211, and the host 300 may be assembled to the milk chamber assembly 200 along the buckling direction of the second buckle portion 201 and the first buckle portion 304.

In an example, as shown in FIG. 13, FIG. 15, and FIG. 16, the milk chamber assembly 200 is provided with a first bearing edge surface 202 spaced apart from the connecting hole 211 in the penetration direction, the host 300 is provided with a second bearing edge surface 320 corresponding to the first bearing edge surface 202, the first bearing edge surface 202 and the second bearing edge surface 320 arch towards a top direction of the breast pump 1a, and the top direction intersects with the penetration direction. The second buckle portion 201 is arranged on the first bearing edge surface 202 and spaced apart from the connecting hole 211 along the penetration direction, and the first buckle portion 304 is arranged on the second bearing edge surface 320. One of the second buckle portion 201 and the first buckle portion 304 is a protrusion whose protruding direction intersects with the penetration direction, and the other one of the second buckle portion 201 and the first buckle portion 304 is a groove whose depth direction intersects with the penetration direction.

When the breast pump 1a is assembled, the protrusion is inserted into the groove along the buckling direction, thereby achieving a buckling connection between the second buckle portion 201 and the first buckle portion 304. Further, the top direction of the breast pump 1a is the buckling direction of the second buckle portion 201 and the first buckle portion 304.

The breast pump accessory 100 includes the soft rubber cover body 110, the heating element 120, and the plug cover plate 130. The heating element 120 is arranged inside the soft rubber cover body 110. The plug cover plate 130 is electrically connected to the heating element 120 and penetrates through the soft rubber cover body 110. The host 300 includes the electrical connection assembly 310. The electrical connection assembly 310 is electrically connected to the plug cover plate 130 to supply power to the heating element 120. The host 300 is provided with the first buckle portion 304 on the periphery of the electrical connection assembly 310. The milk chamber assembly 200 is arranged between the breast pump accessory 100 and the host 300. The milk chamber assembly 200 is provided with the connecting hole 211 whose position corresponds to the plug cover plate 130. The plug cover plate 130 passes through the connecting hole 211 and is connected to the electrical connection assembly 310. The milk chamber assembly 200 is provided with the second buckle portion 201 corresponding to the first buckle portion 304 on the periphery of the connecting hole 211. The second buckle portion 201 is buckled with the first buckle portion 304, so that the heating element 120 has a heating effect. The host 300 is buckled with the milk chamber assembly 200 through the first buckle portion 304 and the second buckle portion 201, so that the host 300 can be relatively fixed with the milk chamber assembly 200, facilitating relative fixation of the breast pump accessory 100 and the milk chamber assembly 200, maintaining stable connection between the electrical connection assembly 310 and the plug cover plate 130, and enabling the heating element 120 to have a stable heating effect.

In an example, the soft rubber cover body 110 is provided with a mounting portion 112 in a protruding manner that corresponds to the connecting hole 211, and the plug cover plate 130 is electrically connected to the heating element 120 and penetrates through the mounting portion 112. The periphery of the mounting portion 112 is provided with a third buckle portion 114. The milk chamber assembly 200 is provided with a fourth buckle portion 212 in the connecting hole 211. The milk chamber assembly 200 is connected to the soft rubber cover body 110, the mounting portion 112 penetrates through the connecting hole 211, and the third buckle portion 114 is buckled with the fourth buckle portion 212 to limit the relative movement of the milk chamber assembly 200 and the soft rubber cover body 110 in a direction of separation from each other.

After the operation of the breast pump 1a, the breast pump accessory 100, the milk chamber assembly 200, and the host 300 can be disassembled, making it easy to clean the breast pump accessory 100 and the milk chamber assembly 200. When the breast pump accessory 100 is cleaned, the soft rubber cover body 110 and the plug cover plate 130 can seal the heating element 120 to prevent the heating element 120 from coming into contact with water, so that the heating element 120 can operate stably. In some examples, the plug cover plate 130 can be fixed to the mounting portion 112 by screws and tightly abut against the mounting portion 112, thereby hindering water from entering the interior of the soft rubber cover body 110 to affect the operation of the heating element 120 when the breast pump accessory 100 is cleaned.

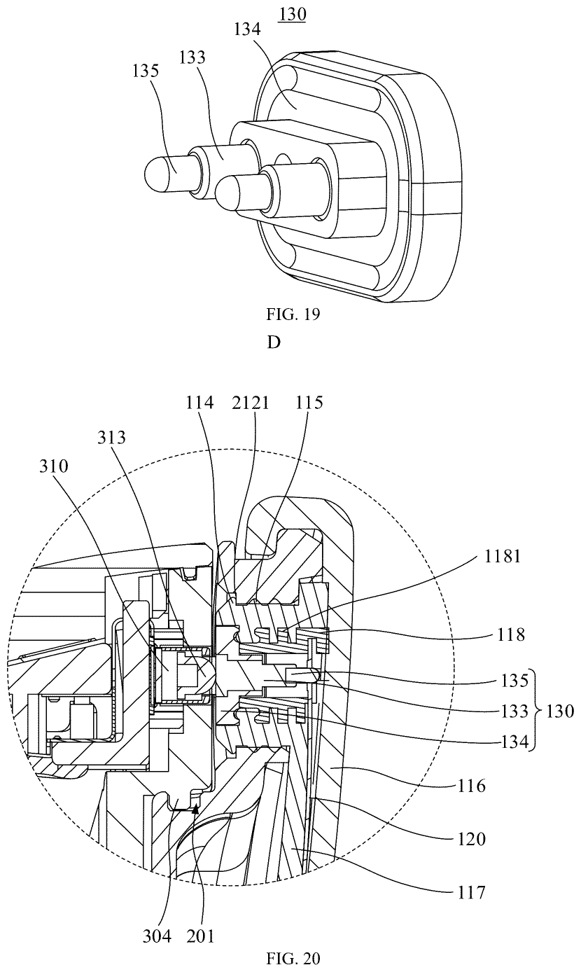

In an example, as shown in FIG. 19 and FIG. 20, the plug cover plate 130 includes a fixing portion 133, a housing 134, and a first elastic movable portion 135. The housing 134 is fixed to the mounting portion 112, the fixing portion 133 is fixedly connected to the housing 134, and the first elastic movable portion 135 is electrically connected to the heating element 120. One end of the fixing portion 133 is exposed relative to the electrical connection assembly 310 and electrically connected to the electrical connection assembly 310, while the other end of the fixing portion 133 is electrically connected to the first elastic movable portion 135. The relative fixation of the housing 134, the mounting portion 112, and the fixing portion 133 is beneficial to sealing between the housing 134 and the mounting portion 112 and between the housing 134 and the fixing portion 133, thereby achieving waterproof protection for the heating element 120. In an example, the fixing portion 133 is a copper pillar.

In an example, as shown in FIG. 20, the electrical connection assembly 310 may include a second elastic movable portion 313. The second elastic movable portion 313 elastically abuts against the plug cover plate 130, so that the electrical connection assembly 310 and the plug cover plate 130 can be stably connected. When the breast pump accessory 100 and the milk chamber assembly 200 are assembled, the second elastic movable portion 313 is compressed by the pressure of the plug cover plate 130, making it easy to assemble the breast pump accessory 100 and the milk chamber assembly 200.

The breast pump accessory 100 and the host 300 are arranged on both sides of the milk chamber assembly 200 separately. The connecting hole 211 formed on the milk chamber assembly 200 allows the electrical connection assembly 310 and the plug cover plate 130 to be connected through the milk chamber assembly 200. Through the buckling connection between the third buckle portion 114 and the fourth buckle portion 212, the mounting portion 112 can be relatively fixed with the milk chamber assembly 200, facilitating stable connection between the electrical connection assembly 310 and the plug cover plate 130.

In some examples, the electrical connection assembly 310 elastically abuts against the plug cover plate 130, where the elastic effect allows the electrical connection assembly 310 and the plug cover plate 130 to have a tendency to move away from each other. The buckling connection between the third buckle portion 114 and the fourth buckle portion 212 can limit the movement of the electrical connection assembly 310 and the plug cover plate 130 away from each other.

In an example, as shown in FIG. 13 and FIG. 17, the third buckle portion 114 is ring-shaped and protrudes from a peripheral wall of the mounting portion 112. The connecting hole 211 includes a first hole segment 2111 and a second hole segment 2112 connected along the penetration direction of the mounting portion 112, a ring-shaped step surface 2121 serving as the fourth buckle portion 212 is formed at the connection of the first hole segment 2111 and the second hole segment 2112, and the ring-shaped step surface 2121 faces away from the soft rubber cover body 110. The third buckle portion 114 is located at the second hole segment 2112 and abuts against the ring-shaped step surface 2121 each other.

In an example, on a cross-section perpendicular to the penetration direction of the mounting portion 112, the size of the second hole segment 2112 is greater than that of the first hole segment 2111, thereby forming the ring-shaped step surface 2121. When the breast pump accessory 100 and the milk chamber assembly 200 are assembled, the third buckle portion 114 passes through the first hole segment 2111 and enters the second hole segment 2112 through elastic deformation. After entering the second hole segment 2112, the third buckle portion 114 deforms and recovers, and abuts against the ring-shaped step surface 2121, and the ring-shaped step surface 2121 can limit the third buckle portion 114 within the second hole segment 2112, to limit the mounting portion 112 within the connecting hole 211, facilitating the stable connection between the electrical connection assembly 310 and the plug cover plate 130.

When the breast pump accessory 100 and the host 300 are disassembled, the third buckle portion 114 can be released from the limitation of the ring-shaped step surface 2121 through elastic deformation, so that the mounting portion 112 can be detached from the connecting hole 211.

In an example, as shown in FIG. 13 and FIG. 17, the peripheral wall of the mounting portion 112 is provided with an elastic supporting protrusion 115 spaced apart from the third buckle portion 114 along the penetration direction of the mounting portion 112, and the elastic supporting protrusion 115 elastically abuts against the hole wall of the connecting hole 211, so that the mounting portion 112 and the connecting hole 211 fit tightly.

Such design can increase the friction between the mounting portion 112 and the hole wall of the connecting hole 211, and limit the detachment of the mounting portion 112 from the connecting hole 211 during the use of the breast pump 1a, facilitating stable operation of the breast pump 1a. On the other hand, the elastic supporting protrusion 115 can play a limiting role, to limit the mounting portion 112 from moving perpendicular to its penetration direction, facilitating stable connection between the electrical connection assembly 310 and the plug cover plate 130.

In an example, as shown in FIG. 13 and FIG. 17, the elastic supporting protrusion 115 is ring-shaped along the circumference of the mounting portion 112, and the elastic supporting protrusion 115 circumferentially abuts against the hole wall of the connecting hole 211.

The ring-shaped elastic supporting protrusion 115 can increase the abutting area between the elastic supporting protrusion 115 and the hole wall of the connecting hole 211, thereby increasing the friction between the mounting portion 112 and the hole wall of the connecting hole 211, to hinder the mounting portion 112 from detaching from the connecting hole 211 during the use of the breast pump 1a.

In some examples, as shown in FIG. 18, the breast pump accessory 100 and the milk chamber assembly 200 abut against each other to enclose a milk storage chamber 213 for storing breast milk. The elastic supporting protrusion 115 at the mounting portion 112 can provide a sealing effect for the milk storage chamber 213.

In an example, as shown in FIG. 17, the quantity of the elastic supporting protrusion 115 is at least two, and the at least two elastic supporting protrusions 115 are spaced apart along the penetration direction of the mounting portion 112. For example, the quantity of the elastic supporting protrusion 115 is two, three, or four.

The at least two elastic supporting protrusions 115 can increase the abutting area between the elastic supporting protrusions 115 and the hole wall of the connecting hole 211, thereby increasing the friction between the mounting portion 112 and the hole wall of the connecting hole 211, and enhancing the limiting effect of the elastic supporting protrusions 115. In some examples, the at least two elastic supporting protrusions 115 can increase the sealing effect on the milk storage chamber 213.

In an example, as shown in FIG. 17, the third buckle portion 114 is an elastic soft rubber portion protruding from the peripheral wall of the mounting portion 112.

As such, the third buckle portion 114 can undergo elastic deformation and deformation recovery, making it easy to assemble the breast pump accessory 100 and the milk chamber assembly 200. Further, the third buckle portion 114 is integrally formed with the peripheral wall of the mounting portion 112, thereby simplifying the manufacturing process for the breast pump accessory 100.

In an example, as shown in FIG. 14 and FIG. 18, the soft rubber cover body 110 includes a second sub cover body 116, a first sub cover body 117, and a plug socket 118. The plug socket 118 is fixed to the second sub cover body 116, the plug cover plate 130 penetrates through the plug socket 118, and the first sub cover body 117 and the second sub cover body 116 are connected through injection molding, hydraulic forming, or silicone injection and further wrap the periphery of the plug socket 118. The plug socket 118 and the first sub cover body 117 wrapping on its periphery form the mounting portion 112. The heating element 120 is arranged between the second sub cover body 116 and the first sub cover body 117, and the fluid channel 111 runs through the second sub cover body 116 and the first sub cover body 117.

Further, the plug socket 118 is provided with an internal mounting hole 113, and the plug cover plate 130 penetrates through the plug socket 118 through the internal mounting hole 113. The internal mounting hole 113 allows the plug cover plate 130 to pass through the soft rubber cover body 110 and be connected to the heating element 120. The plug cover plate 130 can effectively seal the internal mounting hole 113.

The plug cover plate 130 is fixedly connected to the soft rubber cover body 110 through the plug socket 118. The first sub cover body 117 wraps the periphery of the plug socket 118, to achieve connection between the first sub cover body 117 and the plug socket 118 and sealing between the first sub cover body 117 and the plug socket 118, thereby forming waterproof protection for the heating element 120.

In the manufacturing process of the soft rubber cover body 110, the second sub cover body 116 may be first subjected to injection molding, hydraulic forming, or silicone injection molding, then the heating element 120 and the plug socket 118 are assembled on one side of the second sub cover body 116, and finally the first sub cover body 117 is formed on a side of the second sub cover body 116 through injection molding, hydraulic forming, or silicone injection, where the heating element 120 and the plug socket 118 are assembled on the side. When the first sub cover body 117 is formed, the first sub cover body 117 may be connected to the second sub cover body 116, the heating element 120 is located between the second sub cover body 116 and the first sub cover body 117, and the first sub cover body 117 wraps the periphery of the plug socket 118.

In an example, as shown in FIG. 20, the peripheral wall of the plug socket 118 is provided with a tooth-shaped protrusion 1181. The first sub cover body 117 can wrap the tooth-shaped protrusion 1181 during injection molding, hydraulic forming, or silicone injection molding, so that the first sub cover body 117 and the peripheral wall of the plug socket 118 engage with each other to improve the stability and sealing property of connection between the first sub cover body 117 and the plug socket 118.

In an example, the plug cover plate 130 may be fixed to the plug socket 118 by screws. The material of the plug socket 118 is hard plastic or metal, which provides good supporting performance.

In an example, as shown in FIG. 14 and FIG. 18, a heating area of the heating element 120 is arranged around the fluid channel 111.

For example, the heating element 120 may be ring-shaped and fitted over the second sub cover body 116. Such design can improve the uniformity of heating of the soft rubber cover body 110.

In an example, the heating element 120 includes an FPC, i.e., a flexible printed circuit board.

In an example, as shown in FIG. 13 and FIG. 18, the milk chamber assembly 200 is provided with a milk storage chamber 213 or the milk chamber assembly 200 and the soft rubber cover body 110 are enclosed to form a milk storage chamber 213, and a lowest position of the heating area of the heating element 120 in a height direction of the breast pump 1a is higher than or coincides with a preset capacity threshold position of the milk storage chamber 213 in the height direction.

In an example, as shown in FIG. 14, heating circuits are distributed in the heating area of the heating element 120, while heating circuits are not provided in the non-heating area of the heating element 120.

The preset capacity threshold position is a milk level position corresponding to an upper limit of a reasonable milk storage capacity of the milk storage chamber 213. Such arrangement can separate the breast milk from the heating area of the heating element 120, avoiding nutrient loss and bacterial growth caused by prolonged heating of breast milk by the heating element 120.

For example, if the preset capacity threshold position of the milk storage chamber 213 in the height direction is a ½ capacity position, the milk level in the milk storage chamber 213 is often lower than the ½ capacity position in the height direction. Further, if the preset capacity threshold position of the milk storage chamber 213 in the height direction is a ¾ capacity position, the milk level in the milk storage chamber 213 is often lower than the ¾ capacity position in the height direction. Further, if the preset capacity threshold position of the milk storage chamber 213 in the height direction is a ⅘ capacity position, the milk level in the milk storage chamber 213 is often lower than the 4/5 capacity position in the height direction.

The milk chamber assembly 200 and the soft rubber cover body 110 are enclosed to form the milk storage chamber 213, making it easy to clean the milk storage chamber 213 when the milk chamber assembly 200 and the breast pump accessory 100 are disassembled.

In an example, as shown in FIG. 13 and FIG. 18, the milk chamber assembly 200 includes a chamber body 210 and an elastic member 220. The connecting hole 211 is formed in the chamber body 210, the chamber body 210 is connected to the soft rubber cover body 110, and the chamber body 210 is provided with a milk storage chamber 213, or the chamber body 210 and the soft rubber cover body 110 are enclosed to form a milk storage chamber 213. The milk storage chamber 213 is used to store the breast milk sucked by the breast pump 1a.

The connecting hole 211 is spaced apart from the milk storage chamber 213. Further, when the user sits down or lies to use the breast pump 1a, the connecting hole 211 is located above at least a portion of the milk storage chamber 213.

As shown in FIG. 13 and FIG. 18, the chamber body 210 is provided with a transition chamber 214, and the transition chamber 214 has a first opening 215, a second opening 216, and a third opening 217. The transition chamber 214 is connected to the fluid channel 111 through the first opening 215, and the transition chamber 214 is connected to the milk storage chamber 213 through the second opening 216. The elastic member 220 is arranged in the transition chamber 214 and has an expansion state and a contraction state. The host 300 covers the third opening 217 and drives the elastic member 220 to switch between the expansion state and the contracted state through the third opening 217, to change air pressure inside the transition chamber 214.

The transition chamber 214 is exposed relative to the host 300 through the third opening 217, and the elastic member 220 can seal the third opening 217 to isolate the transition chamber 214 from the host 300. The first opening 215 is always in an open state. When the host 300 is used for sucking, the second opening 216 is closed. Meanwhile, the elastic member 220 is in the contraction state through elastic deformation, and the space occupied by the elastic member 220 in the transition chamber 214 is reduced, to decrease the air pressure in the transition chamber 214 and the intake channel, thereby generating suction force to be able to suck breast milk. When the host 300 completes sucking, the second opening 216 is opened, the elastic member 220 is in the expansion state through deformation recovery to increase the air pressure in the transition chamber 214, and the breast milk can flow into the milk storage chamber 213 through the second opening 216.

Further, during the operation of the breast pump 1a, the transition chamber 214 is located above at least a portion of the milk storage chamber 213, thereby facilitating the flow of breast milk into the milk storage chamber 213.

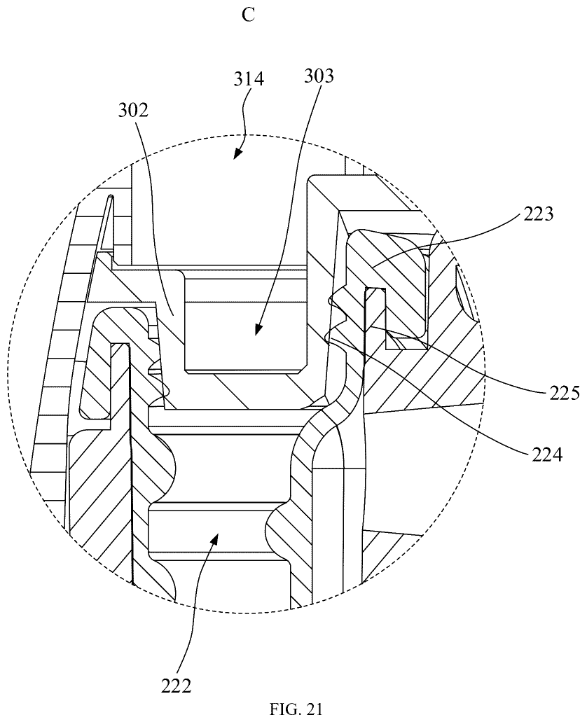

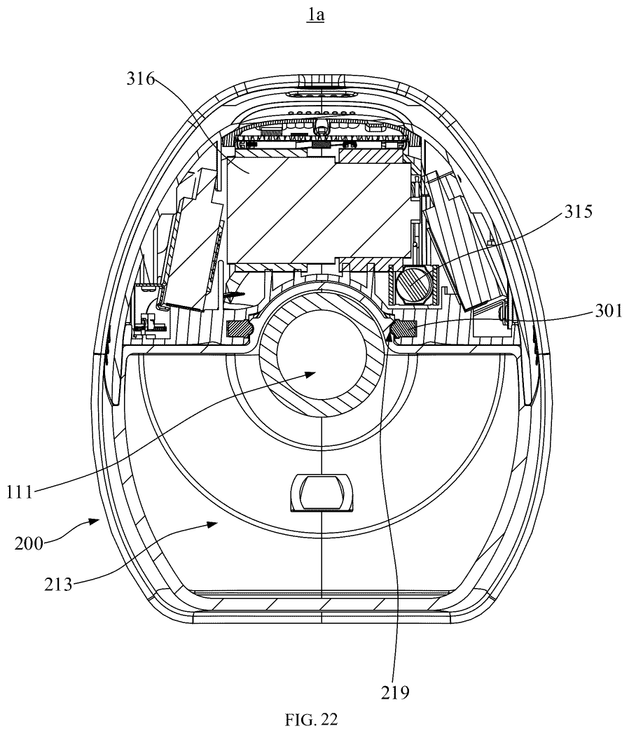

In an example, as shown in FIG. 18 and FIG. 21, the host 300 is provided with a suction chamber 314, or the host 300 and the milk chamber assembly 200 are enclosed to form a suction chamber 314. The elastic member 220 separates the suction chamber 314 from the transition chamber 214, and the host 300 can extract air in the suction chamber 314 through the suction device 316, so that the elastic member 220 is in the contraction state through elastic deformation. The suction chamber 314 may have an air inlet to connect to the outside. The air inlet is blocked when the suction device 316 operates. When the suction device 316 completes air extraction, the air inlet is opened to allow air to enter the suction chamber 314, so that the air pressure inside the suction chamber 314 is recovered, and the elastic member 220 is in the expansion state through deformation recovery.

The elastic member 220 separates the suction chamber 314 from the transition chamber 214, to prevent the host 300 from contaminating the milk in the milk storage chamber 213. When the host 300 is used for sucking, the second opening 216 is closed to limit the air in the milk storage chamber 213 from being sucked into the transition chamber 214, facilitating quick reduction of the air pressure in the transition chamber 214 and the fluid channel 111.

In an example, as shown in FIG. 22, the host 300 may be provided with a solenoid valve 315. When the suction device 316 extracts air, the solenoid valve 315 blocks the air inlet. When the suction device 316 completes air extraction, the solenoid valve 315 opens the air inlet.

In an example, as shown in FIG. 13 and FIG. 18, the milk chamber assembly 200 includes a duckbill valve 230, and the duckbill valve 230 is arranged in the second opening 216 and opened or closed with pressure changes within the transition chamber 214, to close the second opening 216 when the elastic member 220 is in the contraction state and open the second opening 216 when the elastic member 220 is in the expansion state.

The normally-open end of the duckbill valve 230 faces the transition chamber 214, while the other end of the duckbill valve 230 is flexible and selectively opens with air pressure changes within the transition chamber 214. Specifically, the other end of the duckbill valve 230 closes with pressure decrease within the transition chamber 214, thereby closing the second opening 216. When the pressure within the transition chamber 214 recovers, the other end of the duckbill valve 230 opens, thereby opening the second opening 216. The duckbill valve 230 can automatically adjust the opening or closing state of the second opening 216, to adapt to the suction of the host 300.

In an example, as shown in FIG. 13 and FIG. 22, the soft rubber cover body 110 is provided with a channel portion 119 extending and protruding towards one side of the milk chamber assembly 200, the soft rubber cover body 110 is further provided with a fluid channel 111, and the fluid channel 111 runs through the channel portion 119. The milk chamber assembly 200 includes a ring-shaped wall 218 surrounding at least a portion of the periphery of the channel portion 119. The milk chamber assembly 200 is provided with an elastic engagement groove 219 on the periphery of the ring-shaped wall 218, and the host 300 is provided with an elastic bead 301 whose position corresponds to the elastic engagement groove 219. The elastic bead 301 is embedded in the elastic engagement groove 219.

Further, as shown in FIG. 13 and FIG. 22, the soft rubber cover body 110 is provided with a channel portion 119 extending and protruding towards the side of the chamber body 210, and the fluid channel 111 runs through the channel portion 119. The chamber body 210 includes a ring-shaped wall 218 surrounding at least a portion of the periphery of the channel portion 119. The transition chamber 214 is spaced apart from the ring-shaped wall 218, and the first opening 215 is connected to a space enclosed by the ring-shaped wall 218. The storage body 210 is provided with an elastic engagement groove 219 on the periphery of the ring-shaped wall 218, and the host 300 is provided with an elastic bead 301 whose position corresponds to the elastic engagement groove 219. The elastic bead 301 is embedded in the elastic engagement groove 219.

When the breast pump accessory 100 and the milk chamber assembly 200 are assembled, the channel portion 119 can be inserted into the space enclosed by the ring-shaped wall 218, and the first opening 215 can be connected to the fluid channel 111. The ring-shaped wall 218 facilitates the insertion of the channel portion 119 into the milk chamber assembly 200, making it easy to assemble the breast pump accessory 100 and the milk chamber assembly 200.

In an example, the elastic bead 301 includes a bead and a spring that are connected. Through the deformation of the spring, the elastic bead 301 becomes elastic.

The elastic bead 301 is embedded in the elastic engagement groove 219, to limit the loosening of the connected host 300 and milk chamber assembly 200, thereby improving the sucking effect of the host 300. During assembly, the embedding of the elastic bead 301 into the elastic engagement groove 219 can further promote proper assembly of the breast pump accessory 100 and the milk chamber assembly 200 and prompt an assembler to assemble them in place. Further, the elastic engagement groove 219 may be arranged at the end of the ring-shaped wall 218 near the transition chamber 214. Such arrangement is beneficial to improving the stability of connection between the host 300 and the milk chamber assembly 200 at the third opening 217, and reducing the adverse effects of deformation of the elastic member 220 on the stability and sealing property of the connection between the host 300 and the milk chamber assembly 200. In an example, the quantity of the elastic bead 301 may be one, two, or more.

In an example, as shown in FIG. 13 and FIG. 21, the elastic member 220 is provided with a recessed chamber 222 with an open end 221, and the peripheral wall of the elastic member 220 abuts against the chamber body 210. The host 300 has a sealed insertion portion 302, the sealed insertion portion 302 is provided with a through hole 303, and the sealed insertion portion 302 is inserted into the recessed chamber 222 via the open end 221 and sealed against the wall of the recessed chamber 222. The host 300 is used to extract the air inside the recessed chamber 222 via the through hole 303, so as to switch the elastic member 220 between the expansion state and the contraction state.

The peripheral wall of the elastic member 220 abuts against the chamber body 210 and the sealed insertion portion 302, so that the elastic member 220 can effectively block the third opening 217 and the through hole 303, and the air pressure in the transition chamber 214 can respond promptly and quickly to the suction of the host 300. When the host 300 is used to extract air, the air pressure in the recessed chamber 222 decreases, so that the elastic member 220 contracts. When the host 300 completes air extraction, the air pressure in the recessed chamber 222 recovers, so that the elastic member 220 expands. Further, the host 300 is provided with a suction chamber 314, and the suction chamber 314 is connected to the recessed chamber 222 via the through hole 303.

In an example, as shown in FIG. 21, the sealed insertion portion 302 is obliquely frustum-shaped in its extension direction. The size of the sealed insertion portion 302 in a direction perpendicular to the extension direction gradually increases away from a bottom surface of the recessed chamber 222.

Such design can, on the one hand, guide the insertion of the sealed insertion portion 302 into the recessed chamber 222 to facilitate the assembly of the host 300 and the milk chamber assembly 200, and on the other hand, reduce the force of pulling the sealed insertion portion 302 out of the recessed chamber 222 when the host 300 and the milk chamber assembly 200 are disassembled, to reduce the risk of pulling the elastic member 220 out of the transition chamber 214 along with the sealed insertion portion 302.

In an example, as shown in FIG. 13 and FIG. 21, the chamber body 210 is provided with a ring-shaped support wall 225 on the periphery surrounding the third opening 217, the elastic member 220 includes a ring-shaped hanging portion 223 formed by folding outward from the open end 221, and the hanging portion 223 is supported and hung on the support wall 225.

The ring-shaped hanging portion 223 and support wall 225 facilitate the assembly of the elastic member 220 onto the chamber body 210, and also maintain the stability of connection between the elastic member 220 and the chamber body 210.

In an example, as shown in FIG. 13 and FIG. 21, the inner peripheral wall of the recessed chamber 222 is provided with a ring-shaped sealing rib 224, and the ring-shaped sealing rib 224 surrounds and abuts against the sealed insertion portion 302.

The ring-shaped sealing rib 224 can improve the sealing effect between the elastic member 220 and the sealed insertion portion 302, thereby enhancing the working effect of the breast pump 1a.

In an example, as shown in FIG. 16 to FIG. 18, the plug cover plate 130 includes two first power supply contacts 131 and two first temperature measurement contacts 132, and the electrical connection assembly 310 includes two second power supply contacts 311 and two second temperature measurement contacts 312. The two first power supply contacts 131 and the two second power supply contacts 311 are electrically connected in one-to-one correspondence, so that the host 300 supplies power to the heating element 120. The two first temperature measurement contacts 132 and the two second temperature measurement contacts 312 are electrically connected in one-to-one correspondence, so that the host 300 obtains the temperature of the plug cover plate 130.

The two first power supply contacts 131 may be connected to a positive electrode and a negative electrode of a power supply respectively to supply power to the heating element 120. A temperature measurement assembly may be provided in the soft rubber cover body 110. For example, the temperature measurement assembly includes a thermistor, and the temperature measurement assembly can output electrical signals to the host 300 through the two first temperature measurement contacts 132, so that the host 300 obtains the temperature in the soft rubber cover body 110 based on the electrical signals.

Further, as shown in FIG. 13 and FIG. 17, the breast pump accessory 100 may include two plug cover plates 130, and each of the plug cover plates 130 has a first power supply contact 131 and a first temperature measurement contact 132. The plug cover plate 130 may include two fixing portions 133, and the surfaces of the two fixing portions 133 opposite to the electrical connection assembly 310 may serve as the first power supply contact 131 and the first temperature measurement contact 132 respectively.

The host 300 may include two electrical connection assemblies 310, and each of the electrical connection assemblies 310 has a second power supply contact 311 and a second temperature measurement contact 312. The electrical connection assembly 310 may include two second elastic movable portions 313, and the surfaces of the two second elastic movable portions 313 facing the plug cover plate 130 may serve as the second power supply contact 311 and the second temperature measurement contact 312 respectively.

In summary, this example can achieve the buckling connection between the breast pump accessory 100 and the milk chamber assembly 200 through the third buckle portion 114 and the fourth buckle portion 212, enabling the breast pump accessory 100 to be relatively fixed with the milk chamber assembly 200, maintaining a stable connection between the electrical connection assembly 310 and the plug cover plate 130, and enabling the breast pump 1a to have a stable heating effect.

In the description of the present application, the descriptions with reference to the terms “one example”, “example”, “specific example”, or “some examples” mean that the specific features, mechanisms, materials, or characteristics described in conjunction with the example are included in at least one example of the present application. In the description, the schematic expressions of the above terms do not necessarily refer to the same examples. Moreover, the described specific features, mechanisms, materials, or characteristics may be combined in any one or more examples in an appropriate manner. In addition, those skilled in the art can incorporate and combine different examples and features of different examples in the description of this specification on a non-contradictory basis.

Described above are merely the examples of the present application, and the patent scope of the patent application is not limited thereto. Any equivalent structure or equivalent process transformation made through the description and accompanying drawings of the present application, directly or indirectly applied in other related technical fields, is further included in the scope of patent protection of the present application.

What is claimed is:

1. A breast pump comprising:
a soft rubber cover body comprising a fluid channel; and
a heating element comprising a body portion and a connecting portion extending from the body portion, wherein:
the connecting portion comprises a power port,
the soft rubber cover body comprises a mounting hole on an outer wall of the soft rubber cover body away from the fluid channel,
the body portion is arranged in the soft rubber cover body, and the body portion is adapted to the soft rubber cover body,
the connecting portion is arranged in the mounting hole, and the power port is exposed to the soft rubber cover body through the mounting hole, and
the soft rubber cover body is integrally formed with the heating element during injection molding, hydraulic forming, or silicone injection forming.

2. The breast pump of claim 1, wherein:
the body portion is formed by connecting side edges of a curved heating plate in an end-to-end manner,
both the body portion and the connecting portion comprise through holes,
the through holes accommodate injection molding, hydraulic, or silicone injection sol, and
a fixing plate is provided at a connection of the heating element.

3. The breast pump of claim 1, wherein:
the body portion has a frustum shape and is formed by pressing a ring-shaped heating plate,
both the body portion and the connecting portion comprise through holes, and
the through holes accommodate injection molding, hydraulic, or silicone injection sol.

4. The breast pump claim 1, wherein:
the power port comprises a plug socket arranged in the mounting hole,
the connecting portion comprises a contact and a stud, the plug socket is fitted on the stud, and the contact is accommodated in the plug socket.

5. The breast pump of claim 4, wherein:
the power port further comprises a plug cover plate, and the plug cover plate covers the plug socket,
the plug cover plate comprises a spring pin,
the plug socket is accommodated in the mounting hole, and
the spring pin passes through the mounting hole and the plug socket and abuts against the contact.

6. The breast pump of claim 1, wherein:
the soft rubber cover body comprises a first sub cover body and a second sub cover body that are fixedly connected; and
the body portion is arranged between the first sub cover body and the second sub cover body.

7. The breast pump of claim 1, further comprising:
a plug cover plate electrically connected to the heating element, wherein the plug cover plate penetrates through the soft rubber cover body;
a host comprising an electrical connection assembly connected to the plug cover plate to supply power to the heating element, wherein the host comprises a first buckle portion on a periphery of the electrical connection assembly; and
a milk chamber connected to the soft rubber cover body.

8. The breast pump of claim 7, wherein:
the milk chamber comprises a connecting hole,
a position of the connecting hole corresponds to the plug cover plate,
the plug cover plate passes through the connecting hole and is connected to the electrical connection assembly,
the milk chamber comprises a second buckle portion corresponding to the first buckle portion on a periphery of the connecting hole, and
the second buckle portion is buckled with the first buckle portion.

9. The breast pump of claim 8, wherein:
a buckling direction of the second buckle portion and the first buckle portion intersects with a penetration direction of the connecting hole.

10. The breast pump of claim 9, wherein:
the milk chamber comprises a first bearing edge surface spaced apart from the connecting hole in the penetration direction,
the host comprises a second bearing edge surface corresponding to the first bearing edge surface,
the second buckle portion is arranged on the first bearing edge surface and spaced apart from the connecting hole along the penetration direction, and the first buckle portion is arranged on the second bearing edge surface, and
one of the second buckle portion and the first buckle portion is a protrusion, and the other one of the second buckle portion and the first buckle portion is a groove.

11. The breast pump of claim 8, wherein:
the soft rubber cover body comprises a mounting portion in a protruding manner that corresponds to the connecting hole, and the plug cover plate penetrates through the mounting portion,
the periphery of the mounting portion is provided with a third buckle portion,
the milk chamber comprises a fourth buckle portion in the connecting hole,
the mounting portion penetrates through the connecting hole, and
the third buckle portion is buckled with the fourth buckle portion.

12. The breast pump of claim 8, wherein:
the fluid channel is connected to the milk chamber,
the milk chamber comprises a ring-shaped wall surrounding at least a portion of fluid channel,
the milk chamber comprises an elastic engagement groove on a periphery of the ring-shaped wall, and
the host comprises an elastic bead, and the elastic bead is embedded in the elastic engagement groove.

13. The breast pump of claim 8, wherein:
a heating area of the heating element is arranged around the fluid channel, and
the milk chamber comprises a milk storage chamber.

14. A breast pump comprising:
a soft rubber cover assembly comprising a fluid channel, a first cover, and a second cover; and
a heating element arranged between the first cover and the second cover; and
a milk chamber connected to the fluid channel, wherein:
the soft rubber cover assembly comprises a mounting hole on an outer wall of the soft rubber cover body, and
the heating element is adapted to a shape of the soft rubber cover assembly.

15. The breast pump of claim 14, wherein the heating element has a frustum shape.

16. The breast pump of claim 14, wherein heating element comprises a connection portion arranged in the mounting hole.

17. The breast pump of claim 14, wherein the soft rubber cover assembly is integrally formed with the heating element during injection molding, hydraulic forming, or silicone injection forming.

18. The breast pump of claim 14, wherein a heating area of the heating element is arranged around the fluid channel.

19. The breast pump of claim 14, further comprising:
a plug cover plate electrically connected to the heating element, wherein the plug cover plate penetrates through the soft rubber cover assembly; and
a host comprising an electrical connection assembly connected to the plug cover plate to supply power to the heating element, wherein the host comprises a first buckle portion on a periphery of the electrical connection assembly.

20. The breast pump of claim 19, wherein:
the milk chamber comprises a connecting hole,
a position of the connecting hole corresponds to the plug cover plate,
the plug cover plate passes through the connecting hole and is connected to the electrical connection assembly,
the milk chamber comprises a second buckle portion corresponding to the first buckle portion on a periphery of the connecting hole, and
the second buckle portion is buckled with the first buckle portion.
